(19) United States
(12) Patent Application Publication
(10) Pub. No.: US 2014/0329297 A1
(43) Pub. Date: Nov. 6, 2014

(54) Polymeric Thin-Film Tube Connectors, Bioreactors, Systems and Methods

(71) Applicant: Joule Unlimited Technologies, Inc., Bedford, MA (US)

(72) Inventor: John E. Longan, Nashua, NH (US)

(21) Appl. No.: 14/361,973

(22) PCT Filed: Nov. 29, 2012
(86) PCT No.: PCT/US2012/067026

§ 371 Date: May 30, 2014

Related U.S. Application Data
(60) Provisional application No. 61/565,724, filed on Dec. 1, 2011.

(51) Int. Cl. C12M 1/12; F16L 13/10; C12M 1/00

(52) U.S. Cl. CPC C12M 23/06; C12M 23/00; F16L 13/103
USPC 435/252.1; 435/304.1; 435/289.1; 435/420; 435/257.1; 285/285.1; 156/293

(57) ABSTRACT

Polymeric thin-film tube connectors, bioreactors, systems and methods are described, the connectors allowing connection of a polymeric thin-film tube of a bioreactor chamber with a complementary part, typically, rigid port of flange of the bioreactor or bioreactor system. Further, bioreactors having a thin-film reactor chamber and one or more connectors are described. The thin-film reactor chamber can have a thin-film wall for enclosing culture medium and microorganisms. The connector can include a flexible boot. An opening of the thin-film wall couples to the flexible boot. The thin-film wall can extend through a reactor end of the flexible boot and can rest against an interior surface of the flexible boot. The connectors are particularly advantageous for connecting standard utilities in a bioreactor system to thin-film bioreactor chambers or capsules having a thin-film tubular opening.

### RELATED APPLICATION(S)

This application claims the benefit of U.S. Provisional Application No. 61/565,724, filed on Dec. 1, 2011. The entire teachings of the above application(s) are incorporated herein by reference.

### BACKGROUND OF THE INVENTION

One of the primary limitations of using photosynthetic microorganisms as a method of carbon dioxide sequestration or conversion to products has been the need for development of efficient and cost-effective growth systems. This is also the case for closed, controllable systems for the growth of algae and similar organisms.

One of the factors that determine the cost of a closed bioreactor system is reactor chamber cost. Reactor chambers for the growth of microorganisms (including phototrophic microorganisms) face a number of challenges including the risk of contamination with, for example, symbiotic or opportunistic species. Thin-film bioreactor chamber designs can reduce the cost of the reactor chamber. However, thin-film reactor chambers need to be cost-effectively coupled to other parts of a bioreactor system or plant. Specifically, to reduce the cost of using bioreactor chambers, and, particularly, photobioreactor chambers in which one or more thin-film tubular openings are connected to other parts of the bioreactor, for example, a rigid port or pipe of a circulation driver, it is desirable to extend the life-time of the bioreactor chamber as long as possible. As mentioned above, one important factor that reduces the life-time of a bioreactor chamber is contamination. Further, when the bioreactor chamber finally needs to be replaced with a new bioreactor chamber, it is desirable that the replacement can be done easily.

Thus, bioreactors and connectors are needed that facilitate cost effective employment of thin-film bioreactor chambers including reducing labor and cost for the replacement of thin-film enclosures and providing re-usable connectors that limit contamination and are easily cleanable.

### SUMMARY OF THE INVENTION

One embodiment of the present invention is a bioreactor chamber. The bioreactor chamber comprises (i) a polymeric thin-film tube; and (ii) a connector bonded to the polymeric thin-film tube; wherein the connector comprises a polymeric material and has an internal volume with a first opening and a second opening, the second opening being provided by a flexible material, the polymeric thin-film tube, extending through the first opening, is bonded along an entire perimeter of its exterior surface to a surface of the internal volume of the connector between the first opening and the second opening to provide a bonded surface, and the internal volume has a smooth surface between the bonded polymeric thin-film tube and the second opening.

A further embodiment of the present invention is a connector for connecting a polymeric thin-film tube within a bioreactor. The connector comprises (i) a polymeric material; (ii) an internal volume with a smooth surface having a first opening and a second opening, the second opening being provided by a flexible material; wherein the internal volume is adapted to support (a) a polymeric thin-film tube extending through the first opening and (b) bonding along an entire perimeter of an exterior surface of the polymeric thin-film tube to a surface of the internal volume of the connector between the first opening and the second opening to provide a bonded surface; and (iii) one or more rigid collar sections that provide a rigid collar, the rigid collar having a part embedded in the polymeric material of the connector and having a part protruding out of the polymeric material, the embedded part of the rigid collar being dimensioned and positioned such that when the protruding part is pressed against a complementary rigid port, the flexible material forms a seal with the complementary rigid port.

A further embodiment of the present invention is a method of connecting a bioreactor chamber to a rigid port. The method comprises (i) bonding a polymeric thin-film tube of the bioreactor chamber with a connector, the connector being made at least in part of a polymeric material and having an internal volume with a smooth surface having a first opening and a second opening, the second opening being provided by a flexible material; the bonding occurring along an entire perimeter of an exterior surface of the polymeric thin-film tube with a surface of the internal volume of the connector between the first opening and the second opening to provide a bonded surface; and (ii) pressing a protruding part of a rigid collar towards the rigid port, the rigid collar being in part embedded in the polymeric material of the connector and dimensioned and positioned such that when the protruding part is moved towards the rigid port, the flexible material is moved towards the rigid port, with a force sufficient to form a seal between the flexible material and the rigid port along the perimeter of the rigid port; wherein the rigid collar is made from one or more rigid collar sections.

Yet a further embodiment of the present invention is a sealed connection between a polymeric thin-film tube of a bioreactor and a rigid port prepared by bonding a seamless connector with integral gasket with the polymeric thin-film tube and mechanically coupling the seamless connector to form a seal between the gasket and the rigid port.

Another embodiment of the present invention is a bioreactor chamber comprising

(i) a layer of polymeric thin-film tubes; and (ii) a connector bonded to the layer of polymeric thin-film tubes adapted for connection with a rigid port; wherein the connector comprises a polymeric material and has an internal volume with a first opening and a second opening, the second opening being provided by a flexible material, the polymeric thin-film tube, extending through the first opening, is bonded along an entire perimeter of its exterior surface to a surface of the internal volume of the connector between the first opening and the second opening to provide a bonded surface, and the internal volume has a smooth surface between the bonded polymeric thin-film tube and the second opening.

A further embodiment of the present invention is a bioreactor comprising:

a thin-film reactor chamber having a thin-film wall for enclosing culture medium and microorganisms; and a connector comprising a flexible boot wherein the thin-film wall extends through a reactor end of the flexible boot, the thin-film rests against an interior surface of the flexible boot a set length and an opening of the thin-film wall is coupled to the flexible boot.

Yet a further embodiment of the present invention is a bioreactor comprising: a thin-film reactor chamber having a thin-film wall for enclosing culture medium and microorganisms; a circulation driver producing a flow of the culture medium and microorganisms in the thin-film reactor chamber; and a connector coupling the reactor chamber to the circulation driver and comprising a flexible boot wherein the thin-film wall extends through a reactor end of the flexible boot, the thin-film rests against an interior surface of the flexible boot a predetermined set length, an opening of the thin-film wall couples to the flexible boot, and the flexible boot forms a watertight seal against a port of the circulation driver.

A method of producing phototrophic microorganism in a bioreactor comprising the actions of: coupling a thin-film reactor chamber to a circulation driver by compressing a flexible boot against a port of the circulation driver wherein a thin-film wall of the thin-film reactor chamber extends through a reactor end of the flexible boot, the thin-film rests against an interior surface of the flexible boot a set length and an opening of the thin-film wall couples to the flexible boot; and circulating the microorganisms and culture medium through the thin film reactor chamber.

One embodiment of the present invention is a photobioreactor or bioreactor, system, or method thereof. The present invention provides a bioreactor having a thin-film reactor chamber and one or more connectors. The thin-film reactor chamber can have a thin-film wall for enclosing culture medium and microorganisms. The connector can include a flexible boot. An opening of the thin-film wall couples to the flexible boot. The thin-film wall can extend through a reactor end of the flexible boot and can rest against an interior surface of the flexible boot a set length.

Other embodiments can include one or more of the following variations. A clamp can be used to press a connection end of flexible boot to a rigid port. A rigid collar can surround and couple to a connection end of the flexible boot and the clamp can press the rigid collar against a rigid port providing a seal between a connection end of flexible boot and the rigid port. The thin-film wall can be made of a polymeric film. The flexible boot can be made of a cast urethane. An adhesive can be used to couple and provide a seal between the thin-film wall and the flexible boot. The flexible boot can provide greater support on a connection end of the flexible boot than on the reactor end. The flexible boot can increase in thickness and/or rigidity from the reactor end to a connection end of the flexible boot. The flexible boot can have a constant interior diameter along the set length and the constant interior diameter can substantially equal an interior diameter of a rigid port. The thin-film wall can have a thickness of between about 0.002-0.015 inches. The connector produces a watertight and/or airtight seal with the thin-film wall of the reactor chamber and a rigid port.

In yet another embodiment, the bioreactor can have a thin-film reactor chamber, a circulation driver, and a connector. The thin-film reactor chamber can have a thin-film wall for enclosing culture medium and microorganisms. The circulation driver can produce a flow of the culture medium and microorganisms in the thin-film reactor chamber. The connector can couple the reactor chamber to the circulation driver. The connector can include a flexible boot. The thin-film wall can extend through a reactor end of the flexible boot, the thin-film wall can rest against an interior surface of the flexible boot a predetermined set length, an opening of the thin-film wall can couple to the flexible boot, and/or the flexible boot can form a watertight seal against a port of the circulation driver.

A further embodiment of the present invention provides a method of producing phototrophic microorganism in the bioreactor comprising the actions of: coupling a thin-film reactor chamber to a circulation driver by compressing a flexible boot against a port of the circulation driver and circulating the microorganisms and culture medium through the thin-film reactor chamber. A thin-film wall of the thin-film reactor chamber can extend through a reactor end of the flexible boot. The thin-film wall can rest against an interior surface of the flexible boot a set length and an opening of the thin-film wall can couple to the flexible boot.

The present invention is not intended to be limited to a system or method that must satisfy one or more of any stated objects or features of the invention. It is also important to note that the present invention is not limited to the exemplary or primary embodiments described herein. Modifications and substitutions by one of ordinary skill in the art are considered to be within the scope of the present invention.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a profile block diagram of a bioreactor constructed in accordance with an exemplary embodiment of the invention.

FIG. 2 is a cross-sectional schematic view of a thin-film tube of a bioreactor coupled with a connector having a collar, the connector being clamped with a rigid port according to an exemplary embodiment of the invention.

FIG. 3 is a cross-sectional schematic view of a thin-film tube of a bioreactor coupled with a connector clamped with a rigid port according to a second exemplary embodiment of the invention.

FIG. 4 is a cross-sectional schematic view of a thin-film tube of a bioreactor coupled with a connector clamped with a rigid port according to a third exemplary embodiment of the invention.

FIG. 5 shows a cross-sectional schematic view of a polymeric thin-film tube of a bioreactor chamber (not fully shown) coupled to a rigid collar having an integral gasket, according to an exemplary embodiment of the invention.

FIG. 6 shows a perspective schematic view of a connector (here shown without optional fastening devices such as clamps) coupled to channeled polymeric thin-film tubes of a bioreactor chamber (only partially shown), according to an exemplary embodiment of the invention.

FIG. 7 illustrates an exemplary method for coupling a connector according to an exemplary embodiment of the present invention with channeled polymeric thin-film tubes of a bioreactor.

### DETAILED DESCRIPTION OF THE INVENTION

A description of preferred embodiments of the invention follows. The relevant teachings of all patents, published applications and references cited herein are incorporated by reference in their entirety.

The following explanations of terms and methods are provided to better describe the present invention and to guide those of ordinary skill in the art in the practice of the present invention. As used herein, “comprising” means “including” and the singular forms “a” or “an” or “the” include plural references unless the context clearly dictates otherwise. For example, reference to “comprising a phototrophic microorganism” includes one or a plurality of such phototrophic microorganisms. The term “or” refers to a single element of stated alternative elements or a combination of two or more elements, unless the context clearly indicates otherwise.

Unless explained otherwise, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. The materials, methods, and examples are illustrative only and not intended to be limiting. Other features of the invention are apparent from the following detailed description and the claims.

General

Connectors of the present invention allow connecting a polymeric thin-film tube, and particularly, a thin-film tube of a thin-film bioreactor chamber to one or more other chambers or other parts of a bioreactor or bioreactor system. Advantages of the connectors of the present invention include one or more of: reusability, ease of connection and disconnection of thin-film bioreactor chambers, reduction of dead spaces, gaps and the like and associated reduced contamination risk, autoclavability, low-cost, and small pressure drop across the connection established by the connector.

Exemplary embodiments of the invention can provide a connector for a reactor chamber for a photobioreactor. The photobioreactor provides functions of culture containment, photon capture, temperature control, pH control, and CO₂ injection in a highly integrated deign, lowering overall manufacturing, material, and deployment costs. The exemplary embodiments of the invention can facilitate high volume manufacturing and mass deployment with unprecedented scalability. Typically, connections to polymer film bioreactors are permanently welded to the film structure. Exemplary embodiments of the invention can provide re-usable mechanical connections that can have some aseptic aspects to it. The reactor chambers can be pressurized with different fluids, including air, culture, thermal coolant fluid, and flue gas, to create a balance of pressures that can shape the culture layer within the reactor chamber. The presented embodiments can remove the need for more costly integrated systems and/or permanent connections.

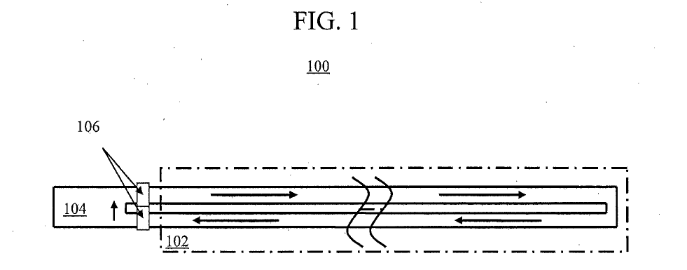

Referring to FIG. 1, an exemplary bioreactor 100 includes a substantially horizontally oriented reactor chamber 102 (here, a thin-film or flexible wall bioreactor chamber) coupled with one or more connectors 106 to a circulation driver 104 or other utility of the bioreactor 100. The circulation driver 104 provides a flow of microorganisms and culture medium in the thin-film or flexible wall bioreactor chamber 102, which is or can be made of translucent or transparent material. The reactor chamber 102 is or can be a thin-film chamber with a high aspect ratio (e.g, thin in cross-sectional view). The culture medium and organism circulates or can circulate through the reactor chamber 102 and maximize exposure via the increased high aspect ratio. After circulating through a loop of the reactor chamber 102, the culture medium and organism exits or can exit through a connector 106 into the circulation driver 104 and back into the entrance of the reactor chamber 102. The reactor chamber 102 is or can be designed in an elongated loop with a path extending away from the circulation driver 104 and returning via a return path parallel from the away path. Embodiments are not limited to one circulation driver or an enclosed loop as shown in FIG. 1. Exemplary embodiments can utilize multiple circulation drivers 104, utilities, and/or comprises or can comprise a single directional path.

Example circulation drivers 104 are not limited to utilizing an induced circulation system, i.e. circulation systems that do not involve active contact with the material being circulated. Exemplary circulating drivers 104 can also utilize active circulation devices that actively apply a contact and apply a force to the circulating material. Example utilities can include, for example but are not limited to, pressure gauges, mixing devices, other reactor chambers, thermal exchangers, storage tanks, and/or sampling devices.

Exemplary embodiments of the invention can provide the reactor chamber 102 for culture containment and productions utilizing various aspects either individually or combined as discussed in the following exemplary embodiments. In one exemplary embodiment, two opposing tapered mating surfaces can be used to encapsulate the full circumference of a thin-film reactor chamber. This connection can then terminate to a sanitary geometry allowing it to connect the bio-reactor to other utilities.

The bioreactors and, particularly, photobioreactors of the present invention can be used for the production of carbon-based products of interest using photoautotroph microorganisms. Further embodiments of the present invention are directed to methods of producing carbon-based products of interest using the bioreactors and, particularly, photobioreactors as described herein. Particular carbon-based products of interest can be fuels. Alternatively, particular carbon-based products of interest include ethanol, propanol, isopropanol, butanol, pentadecane, heptadecane, propane, octane, or diesel.

Connectors

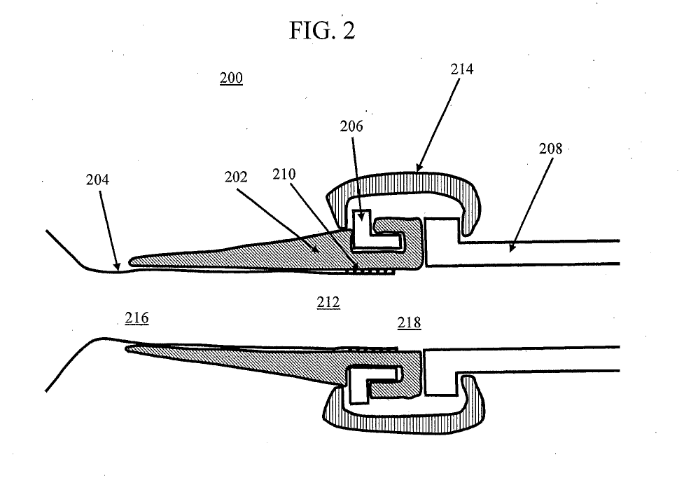

FIG. 2 shows an illustrative sealed connection 200 between a polymeric thin-film tube (or thin-film wall) 204 and a rigid port 208 through a connector including flexible boot 202 that attaches to the exterior of the polymeric thin-film tube (or thin-film wall) 204 in such a way that no additional seams or substantial geometric steps are created. The connector part (e.g., flexible boot) 202 in conjunction with a rigid collar 206 allows for connecting to a rigid port 208 with minimal interruption to the smooth circular space within the connection. Embodiments of the connector 200 can provide a smooth transition from the body of the thin-film wall 204 into the adjoining rigid port 208 with only one appreciable interruption. Embodiments of the connector 200 can generally be used in fluid handling systems to reduce bio-contamination. Embodiments of the connector 200 can provide a sealed connection to the rigid port 208 or similar structure in one processing step. The connector part (e.g., flexible boot) 202 can act as a physical strain relief for the thin-film wall 204 by providing additional support of the thin-film from stress due to the initial flow through the connector 200. This can help prevent the thin-film wall 204 from tearing. The connector part (e.g., flexible boot) 202 can also act as the gasket to create the seal to the rigid port 208 which eliminates the need for an additional gasket or o-ring.

An adhesive bond 210 between the exterior of the thin-film wall 204 and the interior of the connector part (e.g., flexible boot) 202 can be used to attach the thin-film wall 204 to the connector part (e.g., flexible boot) 202 and can provide a seamless, liquid-tight seal. The adhesive bond 210 can be an adhesive tape (such as 3M® 5952 VHB, 3M® transfer tape 9472LE) designed for adhering low surface energy materials. The adhesive bond 210 can also be a variety of chemical adhesive bonds and/or welding type bond.

The connector part 202 is made from a polymeric material and has an internal volume 212 with a first opening 216 and a second opening 218. The second opening is provided by a flexible material and the polymeric thin-film tube 204, extending through the first opening 216, is bonded (e.g., in an adhesive or welded region 210) along an entire perimeter of its exterior surface to a surface of the internal volume 212 of the connector between the first opening 216 and the second opening 218 to provide a bonded surface, and the internal volume has a smooth surface between the bonded polymeric thin-film tube and the second opening.

The rigid collar 206 can be clamped to the rigid port 208 or similar geometry integrated into the rigid piping which compresses the portion of the connector part (e.g. flexible boot) 202 that is positioned between the rigid collar 206 and the rigid port 208 causing it to compress and form a low profile, liquid tight junction between the thin-film wall 204 and the rigid port 208. The thin-film wall 204 (only partially shown) of the reactor chamber (e.g., such as the reactor chamber 102 of FIG. 1) of the photobioreactor is shown to enclose a phototrophic microorganism and culture medium, such as algae or cyanobacteria that flow through a passageway 212 of the connector 200. The thin-film wall 204 of the reactor chamber passes through the reactor end 216 of the connector part 202 and couples to the connector part (e.g. flexible boot) 202. The thin-film wall 204 can extend a set length through the connector part (e.g., flexible boot) 202 to provide support and reduce the strain on the thin-film wall. The connector part (e.g., flexible boot) can increase in rigidity and/or thickness from the reactor end 216 to a connection end 218 of the flexible boot 202. The connector 200 can provide a seal at desired pressures for the reactor chamber (e.g., for a reactor chamber such as 102 in FIG. 1), for example, in the range of 2.0-3.0 psi.

The reactor chamber (e.g., such as reactor chamber 102 in FIG. 1) can be provided by a thin-film material enclosure, typically made from a polymeric material. The phototrophic microorganisms contained in photobioreactors for growth and/or the production of carbon-based products of interest can require light. Therefore, the photobioreactors and, in particular, the reactor chambers are adapted to provide light of a wavelength that is photosynthetically active in the phototrophic microorganism to reach the culture medium. Typically, the thin-film wall 204 of the reactor chamber (e.g., 102) can be transparent for light of a wavelength that is photosynthetically active in the phototrophic microorganism. This can be achieved by proper choice of the material, for example, thin-film material for the reactor chamber to allow light to enter the interior reactor chamber. The thin-film wall 204 of the reactor chamber (e.g. 102) can be a variety of different polymeric films including but not limited to Low-Density PolyEthylene LDPE, nylon reinforced PE, polyester reinforced PE.

In the example shown in FIG. 2, a clamp 214 is used to pull the connector part (e.g., flexible boot) 202 and the rigid collar 206 towards the rigid port 208; however, a variety of fasteners or mechanical devices can be used, for example, a lever, bolt, spring or other device. The rigid collar 206 and/or the rigid port 208 of the connector 200 can be made using common metals, composites, or plastics such as acrylic or acrylonitrile butadiene styrene. Examples of specific materials include but are not limited to stainless steel, Radel® polyphenylsulfone, and Ultem® polyetherimide. The parts can be machined or molded into the desired geometry. The connector part (e.g., flexible boot) 202 can be casted separately or over-molded onto the rigid collar 206 of the connector 200. Embodiments of connector 200 can lend itself to economical mass production using injection molded components; for example, thermoplastic elastomer or silicone can be used for the connector part (e.g., flexible boot) 202 while a wide variety of injection molded rigid plastics can be considered for the rigid collar 206 including polyamide.

The bioreactor (typically, photobioreactor) chambers can be of a variety of different shapes and sizes. The bioreactor size can be influenced by the material and manufacturing choices. For example, in some embodiments of the present invention, the bioreactor chamber is made of a thin film polymeric material which can be, for example, between 1 and 200 meters long with a width of between about 0.2-2 meters. In some embodiments, the reactor chamber 102 is about 40 meters long. A further consideration is transportability of a manufactured photobioreactor, which is greatly enhanced by using flexible thin-film. The reactor chamber 102 can be designed to be folded and/or rolled at least to some extent for more compact storage. For photobioreactors with very large reactor chambers 102 this is a significant advantage, because it can prevent costly transportation permits and oversized transport vehicles, or, alternatively, significant installation costs at the installation site. The connector 200 can be connected to the reactor chamber 102 on site or can be incorporated during manufacturing.

The bioreactor chambers of the present invention include one or more polymeric thin-film tubes, each of which can have a thin-film tubular opening. Typically, an empty (e.g., newly manufactured) bioreactor chamber collapses because the thin-film wall material does not support the shape that it takes on upon inflation with liquid and/or gases (i.e., in a pressurized state). Likewise, typically, the thin-film tubular opening of the empty bioreactor is in a collapsed state prior to connecting a connector of the present invention. In the case of a thin-film bioreactor formed from heat-sealing or otherwise bonding two thin-film sheets to form an enclosure, the thin-film tubular opening can be, for example, provided by a section of the bioreactor in which the two sheets were not bonded. Typically, in this case, the thin-film tubular opening has along its perimeter two edges formed by the bonding process. Preferably, the polymeric thin-film tubes are formed directly as tubes such that above mentioned edges are absent. A further consideration is transportability of a manufactured bioreactor, (e.g., photobioreactor), which is greatly enhanced by using flexible thin-film. The reactor chamber can be designed to be folded and/or rolled at least to some extent for more compact storage. For photobioreactors with very large reactor chambers this is a significant advantage, because it can prevent costly transportation permits and oversized transport vehicles, or, alternatively, significant installation costs at the installation site.

Each reactor chamber of a bioreactor (e.g., photobioreactor) can be a variety of different shapes and dimensions. Typically, however, photobioreactor chambers having channeled polymeric thin-film tubes, typically have channels that are of similar or substantially identical shape and dimensions, for example, channels positioned in parallel with substantially longer channel length than width and substantially the same cross-section area and shape. Various reactor chamber cross-sections are suitable, for example, cylindrical, or half-elliptical. The connector part (e.g., flexible boot 202) can also be a variety of shape including but not limited to, oval, half-elliptical, oblong or rectangular shape. The diameter of the connector part can be designed to match the walls of the reactor chamber, for example, a diameter of between 1 and 12 inches. The passage way 212 of the connector part (e.g., flexible boot) 202 and the rigid port 208 can have closely matching inside diameters to prevent flow obstructions and reduce chamber pressure from dropping across the connection. The connector part (e.g., flexible boot) 202 can be positioned flush with the rigid port 208 to reduce flow obstruction and/or buildup of pathogenic organisms.

The connector can provide a coupling of the reactor chamber 102 to a number of devices that can support the operation of the bioreactors. For example, devices for flowing gases (e.g., carbon dioxide, air, and/or other gases), measurement devices (e.g. optical density meters, thermometers), inlets and outlets, and other elements can be integrated or operationally coupled to the bioreactor. The reactor chambers can include further elements (not shown) such as inlets and outlets, for example, for growth media, carbon sources (e.g., CO₂), and probe devices such as optical density measurement devices and thermometers.

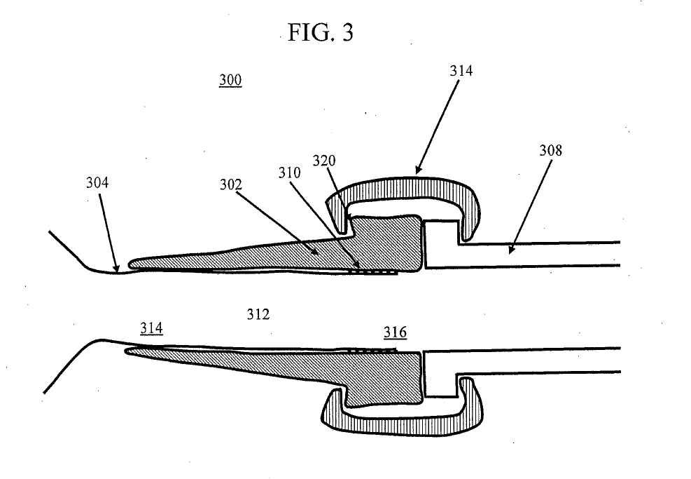

FIG. 3 shows an illustrative sealed connection 300 between a polymeric thin-film tube (or thin-film wall) 304 and a rigid port 308 through a connector including a connector part (e.g., flexible boot) 302. The connector part (e.g., flexible boot) 302 directly couples to a clamp 314. The clamp 314 compresses the portion of the connector part (e.g., flexible boot) 302 against a rigid port 308 causing it to compress and form a low profile, liquid tight junction between a thin-film tube (or wall) 304 and the rigid port 308. A clamp edge 320 of the connector part (e.g., flexible boot) 302 can incorporate a lip or other surface to provide better grip of the clamp 314. As previously described in the prior embodiment, the connector part (e.g., flexible boot) 302 attaches to the exterior of a thin-film wall 304 in such a way that no additional seams or geometric steps are created. The connector part (e.g., flexible boot) 302 can act as a physical strain relief for the thin-film wall 304 by isolating the film from mechanical stress which can help prevent the thin-film wall 304 from tearing. The connector part (e.g., flexible boot) 302 can also act as the gasket to create the seal to the rigid port 308 which eliminates the need for an additional gasket or o-ring. An adhesive bond 310 between the exterior of the thin-film wall 304 and the interior of the flexible boot 302 can be used to attach the thin-film wall 304 to the connector part (e.g., flexible boot) 302 and can provide a seamless, liquid-tight seal. The thin-film wall 304 can extend a set length through the connector part (e.g., flexible boot) 302 to provide support and reduce the strain on the thin-film wall. The connector part (e.g., flexible boot) 302 can increase in rigidity and/or thickness from a reactor end 316 to a connection end 318 of the connector part (e.g., flexible boot) 302. The various components and functions can be provided as previously described in the prior embodiments.

The connector part 302 is made from a polymeric material and has an internal volume 312 with a first opening 314 and a second opening 316. The second opening is provided by a flexible material and the polymeric thin-film tube 304, extending through the first opening 314, is bonded (e.g., in an adhesive or welded region 310) along an entire perimeter of its exterior surface to a surface of the internal volume 312 of the connector between the first opening 314 and the second opening 316 to provide a bonded surface, and the internal volume has a smooth surface between the bonded polymeric thin-film tube and the second opening.

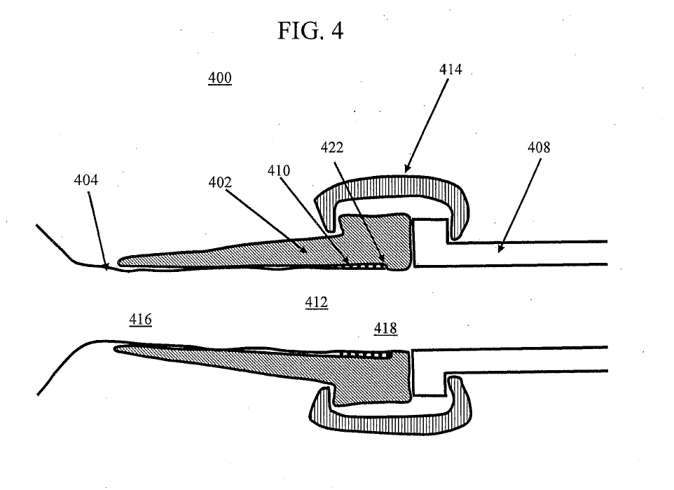

FIG. 4 shows an illustrative sealed connection 400 between a polymeric thin-film tube (or thin-film wall) 404 and a rigid port 408 through a connector including connector part (e.g., flexible boot) 402 that directly couples to a clamp 414. The clamp 414 compresses the portion of the connector part (e.g., flexible boot) 402 against the rigid port 408 causing it to compress and form a low profile, liquid tight junction between a thin-film wall 404 and the rigid port 408. An adhesive bond 410 between the exterior of the thin-film wall 404 and the interior of the connector part (e.g., flexible boot) 402 can be used to attach the thin-film wall 404 to the flexible boot 402 and can provide a seamless, liquid-tight seal. A recessed portion 422 in the interior surface of the connector part (e.g., flexible boot) 402 can be provided to reduce any obstruction of the flow of phototrophic microorganism and culture medium, such as algae or cyanobacteria that flow through a passageway 412 of the connector 400. As previously described in the prior embodiment, the connector part (e.g., flexible boot) 402 can act as a physical strain relief for the thin-film wall 404 by isolating the film from mechanical stress of the flow through the passageway 412. This can help prevent the thin-film wall 404 from tearing. The connector part (e.g., flexible boot) 402 can also act as the gasket to create the seal to the rigid port 408 which eliminates the need for an additional gasket or o-ring. The thin-film wall 404 can extend a set length through the connector part (e.g., flexible boot) 402 to provide support and reduce the strain on the thin-film wall. The connector part (e.g., flexible boot) 402 can increase in rigidity and/or thickness from a reactor end 416 to a connection end 418 of the connector part (e.g., flexible boot) 402. The various components and functions can be provided as previously described in the prior embodiments.

The connector part 402 is made from a polymeric material and has an internal volume 412 with a first opening 416 and a second opening 418. The second opening is provided by a flexible material and the polymeric thin-film tube 404, extending through the first opening 416, is bonded (e.g., in a adhesive or welded region 410) along an entire perimeter of its exterior surface to a surface of the internal volume 412 of the connector between the first opening 416 and the second opening 418 to provide a bonded surface, and the internal volume has a smooth surface between the bonded polymeric thin-film tube and the second opening.

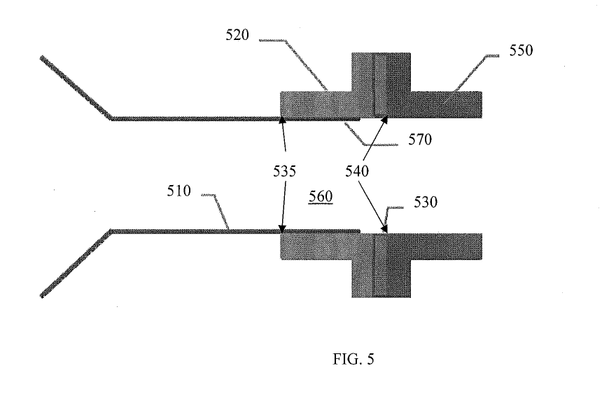

FIG. 5 is a cross-sectional schematic view of a polymeric thin-film tube 510 of a bioreactor chamber (not fully shown) coupled to a rigid collar 520 having an integral gasket 530. The rigid collar has a first opening 535. The integral gasket is made from a flexible material and provides a second opening 540. The integrated gasket is adapted to seal (typically, at liquid pressure of between 0.5 and 5 psi) with a complementary rigid port 550, when the integrated gasket and the rigid port are pressed against each other with a force sufficient to form the seal. The rigid collar 520 and the integral gasket 530 form (part or all of) a connector for connecting the polymeric thin-film tube with the rigid port. The connector includes a polymeric material (the material of the rigid collar and/or the material of the integral gasket) and has an internal volume 560 with a first opening 535 and a second opening 540. The second opening is provided by a flexible material (here, the material of the integral gasket) and the polymeric thin-film tube 510, extending through the first opening 535, is bonded (e.g., in a adhesive or welded region 570) along an entire perimeter of its exterior surface to a surface of the internal volume 560 of the connector between the first opening 535 and the second opening 540 to provide a bonded surface, and the internal volume has a smooth surface between the bonded polymeric thin-film tube and the second opening. The bonded area 570 can extend along the entire internal surface of the rigid collar 520, or it can be a partial area of the internal surface of the rigid collar 520. Further, all or part of the external surface of the polymeric thin-film tube 510 extending through the first opening 535 can be bonded with the rigid collar 520. Typically, the second opening 540 can conform to a substantially circular shape for connection with a complementary part (typically, rigid port) of the bioreactor system.

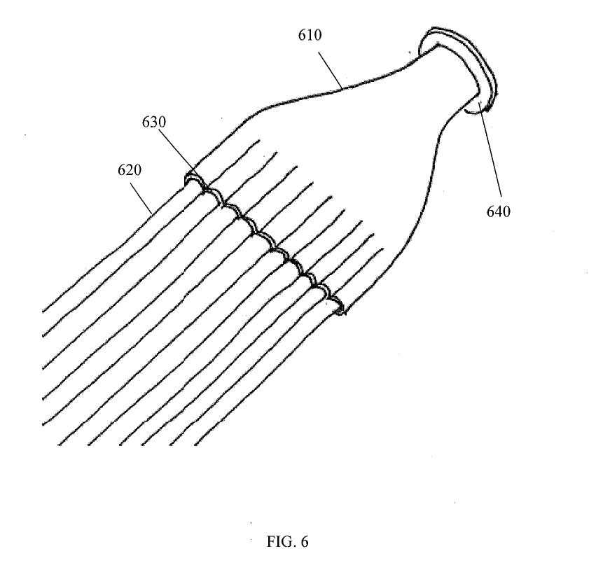

FIG. 6 is a perspective schematic view of a connector 610 (here shown without optional fastening devices such as clamps) coupled to channeled polymeric thin-film tubes 620 (e.g., a plurality of typically horizontally layered polymeric thin-film tubes) of a bioreactor chamber (only partially shown). The connector has an internal volume with a first opening 630 that is adapted to conform to the shape of the polymeric thin-film tubes (here shown in pressurized shape). The connector further includes a second opening (not visable in the perspective view) provided by a flexible material. The flexible material allows formation of a sealed connection with a complementary part (typically, rigid port) of the bioreactor system. The flexible material can be provided by an integral gasket. Further, the connector includes a protruding part 640 for fastening of the connector with the complementary part. The polymeric thin-film tubes 620, extending through the first opening 630, are bonded along an entire perimeter of their exterior surface to a surface of the internal volume of the connector between the first opening 630 and the second opening to provide a bonded surface, and the internal volume has a smooth surface between the bonded polymeric thin-film tube and the second opening. The bonded area can extend along the entire internal surface of the connector, or it can be a partial area of the internal surface of the connector. Further, all or part of the external surface of the polymeric thin-film tubes 620 extending through the first opening 630 can be bonded with the connector 610. Typically, the second opening can conform to a substantially circular shape for connection with a complementary part (typically, rigid port) of the bioreactor system.

The connectors of the present invention can include a flexible boot and/or a rigid collar. The flexible boot can be made, for example, with cast urethane, and the rigid collar can be machined from a rigid plastic or metal, and formed using SLA, laser sintering or some other 3D printing technique. A two sided adhesive tape (such as 3M 5952 VHB or 3M transfer tape 9472LE) designed for adhering to low surface energy materials can be used to affix the polymeric thin-film tube(s) to the flexible boot.

The connectors of the present invention lend themselves to economical mass production using injection molded components; thermoplastic elastomer or silicone may be used for a flexible boot element (if present) while a wide variety of injection molded rigid plastics can be considered for a rigid collar (if present) including polyamide. Two sided adhesive tape as described in the previous paragraph is a viable option for attaching the polymeric thin-film tube(s) to the inside of the connector for production quantities but other attachment methods may also be considered such as heat, spin welding or ultrasonic welding.

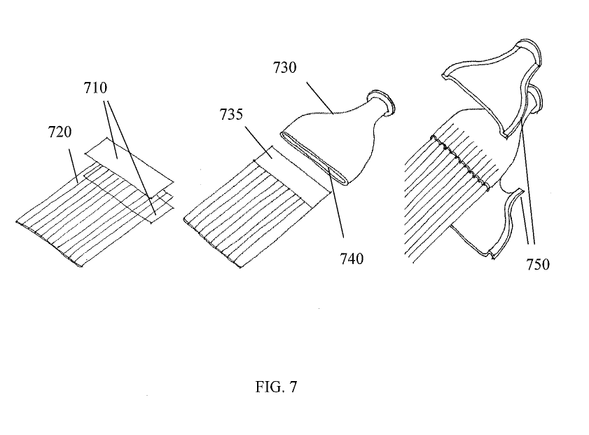

FIG. 7 illustrates an exemplary method for coupling a connector according to an exemplary embodiment of the present invention with channeled polymeric thin-film tubes of a bioreactor. Firstly, as shown on the left-hand side, a two sided adhesive tape 710 is bonded with the unpressurized (i.e., substantially empty, and, thus, flat) channeled polymeric thin-film tubes 720 such that the tape is bonded along an entire perimeter of the exterior surface of the channeled polymeric thin-film tubes. Secondly, as illustrated in the next two drawings (middle and right-had side), the connector 730 and the unpressurized channeled polymeric thin-film tubes 720 are bonded by coupling the second side of the adhesive tape 735 (only the top side is visible in this perspective view) with an internal surface 740 of the connector. On the right-hand side the thin-film tubes are illustrated in pressurized (e.g., with liquid and/or gas) state. The adhesive tape and typically at least the material of the connector in the bonded area is sufficiently flexible to conform to the pressurized shape of the channeled polymeric thin-film tubes. A rigid (e.g., two part) shell 750 can be coupled with the connector to add support.

The connectors of the present invention can provide a seal at internal pressures sufficient for operation of the bioreactor chamber, for example, in the range of 0.1-5.0 psi, more typically, 0.1 to 2.0 psi.

Generally, the connectors of the present invention can connect polymeric thin-film tubes of a bioreactor with a complementary part (typically, a rigid port) of the bioreactor system. However, more generally, the connectors of the present invention can be connected to thin-film tubes of any type of enclosure, and, accordingly, can have uses outside the bioreactor field.

Generally, the connectors (and, typically, the first connector part) of the present invention have a first side with a first opening into an internal volume and a second side with a second opening into the internal volume, thereby allowing gas and/or liquid flow through the connector. However, the connectors of the present invention can also have more than two openings, for example, a T-shaped connector, or a cross-shaped connector. Each opening of the connector can be adapted for connecting to a polymeric thin-film tube (i.e., any of the above described embodiments) or be adapted for connecting with a complementary part (typically, rigid port) of the bioreactor system.

The polymeric thin-film tubes can be, but are not limited to, polymers such as low density polyethylene, nylon reinforced polyethylene, and polyester reinforced polyethylene.

Typically, when the polymeric thin-film tube is substantially empty (i.e., not pressurized with liquid and/or gas) the polymeric thin-film tube is substantially flat.

The connectors of the present invention can include one or more polymeric materials, each of which can have a different rigidity; however, the connectors include at least one flexible material adapted for forming a seal with a complementary part of a bioreactor or bioreactor system. Suitable polymeric materials include, but are not limited to polyamide, high density polyethylene, polyvinyl chloride, thermoplastic elastomer and silicone. Suitable flexible materials include, but are not limited to thermoplastic elastomer and silicone. In certain embodiments of the present invention, the connector (excluding any fastening devices) is made of one polymeric flexible material.

In other embodiments, the connector includes two different polymeric flexible materials, one for bonding with the polymeric thin-film tube and one polymeric flexible material adapated for forming a seal with a complementary part of a bioreactor or bioreactor system.

In yet other embodiments, the connector includes a rigid polymeric material for bonding with the polymeric thin-film tube and one polymeric flexible material adapated for forming a seal with a complementary part of a bioreactor or bioreactor system.

In yet further embodiments, the connector includes a rigid collar made from a polymeric material, The polymeric thin-film tube can be directly bonded to an inside surface of the rigid collar, or the polymeric thin-film tube can be bonded to an inside surface of a second polymeric material which is coupled to the rigid collar (e.g., the rigid collar can be embedded into the second polymeric material) and the connector includes a flexible polymeric material adapated for forming a seal with a complementary part of a bioreactor or bioreactor system.

The connector of the present invention can provide a smooth interior transition from the polymeric thin-film tube to the complementary part (typically, rigid port) of a bioreactor or bioreactor system. Typically, the sealed connection has only one material step or seam thereby reducing potential bio-contamination.

Photobioreactor Biomass Productivity

The connector for the photobioreactor also provides methods to achieve organism productivity as measured by production of desired products, which includes cells themselves.

The desired level of products produced from the engineered light capturing organisms in the solar biofactory system can be of commercial utility. For example, the engineered light capturing organisms in the solar biofactory system convert light, water and carbon dioxide to produce fuels, biofuels, biomass or chemicals at about 5 to about 10 g/m2/day, in certain embodiments about 15 to about 42 g/m²/day and in more preferred embodiments, about 30 to 45 g/m²/day or greater.

The photobioreactor system affords high areal productivities that offset associated capital cost. Superior areal productivities are achieved by: optimizing cell culture density through control of growth environment, optimizing CO₂ infusion rate and mass transfer, optimizing mixing to achieve highest photosynthetic efficiency/organisms, achieving maximum extinction of insolating light via organism absorption, and achieving maximum extinction of C0₂ and initial product separation.

In particular, the southwestern U.S. has sufficient solar insolation to drive maximum areal productivities to achieve about >25,000 gal/acre/year ethanol or about >15,000 gal/acre/year diesel, although a majority of the U.S. has insolation rates amenable to cost effective production of commodity fuels or high value chemicals.

Furthermore, CO₂ is also readily available in the southwestern U.S. region, which is calculated to support large scale commercial deployment of the invention to produce 25-70 g/m²/day ethanol, or 70 Bn gal/year diesel.

### DEFINITIONS

Suitable phototrophic microorganisms can produce a carbon-based product and/or the phototrophic microorganism itself can be processed as feed stock for the production of a carbon-based product. Particularly suitable phototrophic microorganisms can be genetically engineered to produce a desired carbon-based product. Exemplary suitable phototrophic microorganisms are described in U.S. Pat. No. 7,919,303, U.S. Pat. No. 7,794,969, U.S. patent application Ser. No. 12/833,821, U.S. patent application Ser. No. 13/054,470, U.S. patent application Ser. No. 12/867,732, WO/2009/111513, WO/2009/036095, WO/2011/005548, WO/2011/006137 and WO/2011/011464.

“Carbon-based products of interest” include alcohols such as ethanol, propanol, isopropanol, butanol, fatty alcohols, fatty acid esters, ethyl esters, wax esters; hydrocarbons and alkanes such as pentadecane, heptadecane, propane, octane, diesel, Jet Propellant 8 (JP8); polymers such as terephthalate, 1,3-propanediol, 1,4-butanediol, polyols, Polyhydroxyalkanoates (PHA), poly-beta-hydroxybutyrate (PHB), acrylate, adipic acid, ε-caprolactone, isoprene, caprolactam, rubber; commodity chemicals such as lactate, docosahexaenoic acid (DHA), 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-aminodeacetoxycephalosporanic acid (7-ADCA)/cephalosporin, erythromycin, polyketides, statins, paclitaxel, docetaxel, terpenes, peptides, steroids, omega fatty acids and other such suitable products of interest. Such products are useful in the context of biofuels, industrial and specialty chemicals, as intermediates used to make additional products, such as nutritional supplements, neutraceuticals, polymers, paraffin replacements, personal care products and pharmaceuticals. More typical carbon-based products are fuels (e.g. alcohols or alkanes). Even more typically, carbon-based products are ethanol, propanol, isopropanol, butanol, terpenes, alkanes such as pentadecane, heptadecane, octane, propane, fatty acids, fatty esters, fatty alcohols, olefins or diesel.

As used herein, “light of a wavelength that is photosynthetically active in the phototrophic microorganism” refers to light that can be utilized by the microorganism to grow and/or produce carbon-based products of interest, for example, fuels including biofuels.

As used herein, “transparent” refers to an optical property that allows passage of light of a wavelength that is photosynthetically active in the phototrophic microorganism and or other desirable wavelengths of light.

As used herein, “flexible wall” refers to a sheet or sheets of material that have the ability to flex or bend under a relative force or pressure is applied to a surface during operation.

As used herein, “thin-film” refers to a flexible film, for example, a polymer or polymer composite film. Thickness of the film or sheet can be less than 500 micrometers, preferable from 100 to 200 micrometers.

“Phototrophs” or “photoautotrophs” are organisms that carry out photosynthesis such as, eukaryotic plants, algae, protists and prokaryotic cyanobacteria, green-sulfur bacteria, green non-sulfur bacteria, purple sulfur bacteria, and purple non-sulfur bacteria. Phototrophs include natural and engineered organisms that carry out photosynthesis and hyperlight capturing organisms.

The photobioreactors of the present invention are adapted to support a biologically active environment that allows chemical processes involving photosynthesis in organisms such as phototrophic organisms to be carried out, or biochemically active substances to be derived from such organisms. The photobioreactors can support aerobic or anaerobic organisms.

As used herein, “organisms” encompasses autotrophs, phototrophs, heterotrophs, engineered light capturing organisms and at the cellular level, e.g., unicellular and multicellular.

A “spectrum of electromagnetic radiation” as used herein, refers to electromagnetic radiation of a plurality of wavelengths, typically including wavelengths in the infrared, visible and/or ultraviolet light. The electromagnetic radiation spectrum is provided by an electromagnetic radiation source that provides suitable energy within the ultraviolet, visible, and infrared, typically, the sun.

A “biosynthetic pathway” or “metabolic pathway” refers to a set of anabolic or catabolic biochemical reactions for converting (transmuting) one chemical species into another. For example, a hydrocarbon biosynthetic pathway refers to the set of biochemical reactions that convert inputs and/or metabolites to hydrocarbon product-like intermediates and then to hydrocarbons or hydrocarbon products.

Anabolic pathways involve constructing a larger molecule from smaller molecules, a process requiring energy. Catabolic pathways involve breaking down of larger molecules, often releasing energy.

As used herein, “light” generally refers to sunlight but can be solar or from artificial sources including incandescent lights, LEDs fiber optics, metal halide, neon, halogen and fluorescent lights.

The foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of this invention are presented for purposes of illustration and description. They are not intended to be exhaustive or to limit the invention to the precise forms disclosed; obviously many modifications and variations are possible in view of the above teachings. The embodiments were chosen and described in order to best explain the principles of the invention and its practical applications. These procedures will enable others, skilled in the art, to best utilize the invention and various embodiments with various modifications. It is intended that the scope of the invention be defined by the following claims and their equivalents. Modifications and substitutions by one of ordinary skill in the art are considered to be within the scope of the present invention, which is not to be limited except by the following claims.

While this invention has been particularly shown and described with references to example embodiments thereof, it will be understood by those skilled in the art that various changes in form and details can be made therein without departing from the scope of the invention encompassed by the appended claims.

The invention claimed is:

1. A bioreactor chamber comprising
(i) a polymeric thin-film tube; and
(ii) a connector bonded to the polymeric thin-film tube; wherein the connector comprises a polymeric material and has an internal volume with a first opening and a second opening, the second opening being provided by a flexible material, the polymeric thin-film tube, extending through the first opening, is bonded along an entire perimeter of its exterior surface to a surface of the internal volume of the connector between the first opening and the second opening to provide a bonded surface, and the internal volume having a smooth surface between the bonded polymeric thin-film tube and the second opening.

2. The bioreactor chamber of claim 1, further comprising one or more rigid collar sections that provide a rigid collar, the rigid collar having a part embedded in the polymeric material of the connector and having a part protruding out of the polymeric material, the embedded part of the rigid collar being dimensioned and positioned such that when the protruding part is pressed against a complementary rigid port, the flexible material forms a seal with the complementary rigid port.

3. The bioreactor chamber of claim 1 or 2, wherein the connector is made at least in part from a second flexible material between the first opening and the bonded surface.

4. The bioreactor chamber of any one of the preceding claims, comprising a layer of connected polymeric thin-film tubes, wherein the polymeric thin-film tube is one of the connected polymeric thin-film tubes.

5. The bioreactor chamber of claim 4, wherein the connector is made from a second flexible material for a set length from the first opening, and the layer of connected polymeric thin-film tubes extends through the first opening and is bonded along an entire perimeter of its exterior surface to a surface of the internal volume provided by the second flexible material.

6. The bioreactor chamber of any of the preceding claims, wherein the polymeric thin film tube is surrounded by a part of the connector and the polymeric thin-film tube is not bonded in said part.

7. The bioreactor chamber of any of the preceding claims, wherein the polymeric material and the flexible material are the same material.

8. The bioreactor chamber of any of the preceding claims, wherein the polymeric material and the second flexible material are the same material.

9. The bioreactor chamber of any of the preceding claims, wherein the connector increases in thickness from the first opening and towards the second opening.

10. The bioreactor chamber of claim 1, 5 or 9, wherein the first opening and the second opening are opposing openings.

11. The bioreactor chamber of claim 1, wherein the internal volume has a substantially constant interior diameter.

12. A connector for connecting a polymeric thin-film tube within a bioreactor, comprising
(i) a polymeric material;
(ii) an internal volume with a smooth surface having a first opening and a second opening, the second opening being provided by a flexible material; wherein the internal volume is adapted to support (a) a polymeric thin-film tube extending through the first opening and (b) bonding along an entire perimeter of an exterior surface of the polymeric thin-film tube to a surface of the internal volume of the connector between the first opening and the second opening to provide a bonded surface; and (iii) one or more rigid collar sections that provide a rigid collar, the rigid collar having a part embedded in the polymeric material of the connector and having a part protruding out of the polymeric material, the embedded part of the rigid collar being dimensioned and positioned such that when the protruding part is pressed against a complementary rigid port, the flexible material forms a seal with the complementary rigid port.

13. The connector of claim 12, wherein the connector is made at least in part from a second flexible material between the first opening and the bonded surface.

14. The connector of claim 12, wherein the connector is made from a second flexible material for a set length from the first opening, and the polymeric thin-film tube is part of a layer of connected polymeric thin-film tubes.

15. The connector of any one of claims 12-14, wherein the polymeric material and the flexible material are the same material.

16. The connector of any one of claims 12-15, wherein the polymeric material and the second flexible material are the same material.

17. The connector of any one of claims 12-16, wherein the connector increases in thickness from the first opening and towards the second opening.

18. The connector of any one of claims 12-17, wherein the first opening and the second opening are opposing openings.

19. The connector of any one of claims 12-18, wherein the internal volume has a substantially constant interior diameter which substantially equals an interior diameter of the rigid port.

20. A method of connecting a bioreactor chamber to a rigid port, comprising (i) bonding a polymeric thin-film tube of the bioreactor chamber with a connector, the connector being made at least in part of a polymeric material and having an internal volume with a smooth surface having a first opening and a second opening, the second opening being provided by a flexible material; the bonding occurring along an entire perimeter of an exterior surface of the polymeric thin-film tube with a surface of the internal volume of the connector between the first opening and the second opening to provide a bonded surface; and
(ii) pressing a protruding part of a rigid collar towards the rigid port, the rigid collar being in part embedded in the polymeric material of the connector and dimensioned and positioned such that when the protruding part is moved towards the rigid port, the flexible material is moved towards the rigid port, with a force sufficient to form a seal between the flexible material and the rigid port along the perimeter of the rigid port;
wherein the rigid collar is made from one or more rigid collar sections.

21. The method of claim 20, further comprising extending the polymeric thin film tube through the first opening of the connector.

22. The method of claim 20, wherein the polymeric thin-film tube is one of a plurality of connected polymeric thin-film tubes positioned in a layer.

23. The method of claim 20 or 21, further comprising clamping the protruding part of the rigid collar with the rigid port to thereby press the protruding part of a rigid collar towards the rigid port.

24. The bioreactor chamber of any one of claims 1-11, wherein part of the polymeric thin-film tube rests against part of the surface of the internal volume of the connector.

25. A sealed connection between a polymeric thin-film tube of a bioreactor and a rigid port prepared by bonding a seamless connector with integral gasket with the polymeric thin-film tube and mechanically coupling the seamless connector to form a seal between the gasket and the rigid port.

26. A bioreactor chamber comprising
(i) a layer of polymeric thin-film tubes; and
(ii) a connector bonded to the layer of polymeric thin-film tubes adapted for connection with a rigid port; wherein the connector comprises a polymeric material and has an internal volume with a first opening and a second opening, the second opening being provided by a flexible material, the polymeric thin-film tube, extending through the first opening, is bonded along an entire perimeter of its exterior surface to a surface of the internal volume of the connector between the first opening and the second opening to provide a bonded surface, and the internal volume has a smooth surface between the bonded polymeric thin-film tube and the second opening.

27. A bioreactor comprising:
a thin-film reactor chamber having a thin-film wall for enclosing culture medium and microorganisms; and
a connector comprising a flexible boot wherein the thin-film wall extends through a reactor end of the flexible boot, the thin-film rests against an interior surface of the flexible boot a set length and an opening of the thin-film wall is coupled to the flexible boot.

28. The bioreactor of claim 27, further comprising a clamp wherein the clamp presses a connection end of flexible boot to a rigid port.

29. The bioreactor of claim 27 or 28, further comprising a rigid collar surrounding and/or embedded in a connection end of the flexible boot, and coupled to the connection end of the flexible boot.

30. The bioreactor of claim 27, further comprising a rigid collar surrounding and/or embedded in a connection end of the flexible boot, and coupled to the connection end of the flexible boot, and a clamp wherein the clamp presses the rigid collar towards a rigid port providing a seal between a connection end of flexible boot and the rigid port.

31. The bioreactor of any one of the preceding claims, wherein the thin-film wall is coupled to the interior surface of the flexible boot.

32. The bioreactor of claim 27, wherein the thin-wall is made of a polymeric film.

33. The bioreactor of claim 27, wherein the flexible boot is made of a cast urethane.

34. The bioreactor of claim 27, wherein an adhesive couples and provides a seal between the thin-film wall and the flexible boot.

35. The bioreactor of claim 27, wherein the flexible boot provides greater support on a connection end of the flexible boot than on the reactor end.

36. The bioreactor of claim 27, wherein the flexible boot increases in thickness from the reactor end to a connection end of the flexible boot.

37. The bioreactor of claim 27, wherein the flexible boot increases in rigidity from the reactor end to a connection end of the flexible boot.

38. The bioreactor of claim 27, wherein the flexible boot has a constant interior diameter along the set length.

39. The bioreactor of claim 27, wherein the flexible boot has a constant interior diameter along the set length and the constant interior diameter substantially equals an interior diameter of a rigid port.

40. The bioreactor of claim 27, wherein the thin-film wall has a thickness of between about 0.002-0.015 inches.

41. The bioreactor of claim 27, wherein the connector produces a watertight seal with the thin-film wall of the reactor chamber and a rigid port.

42. The bioreactor of claim 27, wherein the flexible boot has an oval shape to receive the thin-film walls of the reactor chamber.

43. A bioreactor comprising:
a thin-film reactor chamber having a thin-film wall for enclosing culture medium and microorganisms;
a circulation driver producing a flow of the culture medium and microorganisms in the thin-film reactor chamber; and
a connector coupling the reactor chamber to the circulation driver and the comprising a flexible boot wherein the thin-film wall extends through a reactor end of the flexible boot, the thin-film rests against an interior surface of the flexible boot a predetermined set length, an opening of the thin-film wall couples to the flexible boot, and the flexible boot forms a watertight seal against a port of the circulation driver.

44. The bioreactor of claim 43, further comprising a clamp wherein the clamp presses a connection end of flexible boot to the port.

45. The bioreactor of claim 43 or 44, further comprising a rigid collar surrounding and/or embedded in a connection end of the flexible boot, and coupled to the connection end of the flexible boot.

46. The bioreactor of claim 17, further comprising a rigid collar surrounding and/or embedded in a connection end of the flexible boot, and coupled to the connection end of the flexible boot and a clamp wherein the clamp presses the rigid collar towards the port providing a seal between a connection end of flexible boot and the port.

47. The bioreactor of anyone of claims 43-46, wherein the thin-film wall couples to the interior surface of the flexible boot.

48. The bioreactor of claim 43, wherein the thin-wall is made of a polymeric film.

49. The bioreactor of claim 43, wherein the flexible boot is made of a cast urethane.

50. The bioreactor of claim 43, wherein an adhesive couples and provides a seal between the thin-film wall and the flexible boot.

51. The bioreactor of claim 43, wherein the flexible boot provides greater support on a connection end of the flexible boot than on the reactor end.

52. The bioreactor of claim 43, wherein the flexible boot increases in thickness from the reactor end to a connection end of the flexible boot.

53. The bioreactor of claim 43, wherein the flexible boot increases in rigidity from the reactor end to a connection end of the flexible boot.

54. The bioreactor of claim 43, wherein the flexible boot has a constant interior diameter along the set length.

55. The bioreactor of claim 43, wherein the flexible boot has a constant interior diameter along the set length and the constant interior diameter substantially equals an interior diameter of a rigid port.

56. The bioreactor of claim 43, wherein the thin-film wall has a thickness of between about 0.002-0.015 inches.

57. The bioreactor of claim 43, wherein the flexible boot has an oval shape to receive the thin-film walls of the reactor chamber.

58. A method of producing phototrophic microorganism in the bioreactor comprising:
coupling a thin-film reactor chamber to a circulation driver by compressing a flexible boot against a port of the circulation driver wherein a thin-film wall of the thin-film reactor chamber extends through a reactor end of the flexible boot, the thin-film rests against an interior surface of the flexible boot a set length and an opening of the thin-film wall couples to the flexible boot; and
circulating the microorganisms and culture medium through the thin film reactor chamber.

59. The method of producing phototrophic microorganism in the bioreactor of claim 58, wherein coupling action further comprises activating a clamp that presses a connection end of the flexible boot to a rigid port.

60. The method of producing phototrophic microorganism in the bioreactor of claim 58, wherein the flexible boot further comprising a rigid collar surrounding and/or embedded in a connection end of the flexible boot, and coupled to the connection end of the flexible boot.

61. The method of producing phototrophic microorganism in the bioreactor of claim 58, wherein coupling action further comprises activating a clamp that presses a rigid collar surrounding and/or embedded in a connection end of the flexible boot, and coupled to the connection end of the flexible boot and a clamp against a rigid port providing a seal between a connection end of flexible boot and the rigid port.

62. The method of producing phototrophic microorganism in the bioreactor of claim 58, wherein the thin-film wall couples to the interior surface of the flexible boot.

63. The method of producing phototrophic microorganism in the bioreactor of claim 58, wherein the thin-wall is made of a polymeric film.

64. The method of producing phototrophic microorganism in the bioreactor of claim 58, wherein the flexible boot is made of a cast urethane.

65. The method of producing phototrophic microorganism in the bioreactor of claim 58, wherein an adhesive couples and provides a seal between the thin-film wall and the flexible boot.

66. The method of producing phototrophic microorganism in the bioreactor of claim 58, wherein the flexible boot provides greater support on a connection end of the flexible boot than on the reactor end.

67. The method of producing phototrophic microorganism in the bioreactor of claim 58, wherein the flexible boot increases in thickness from the reactor end to a connection end of the flexible boot.

68. The method of producing phototrophic microorganism in the bioreactor of claim 58, wherein the flexible boot increases in rigidity from the reactor end to a connection end of the flexible boot.

69. The method of producing phototrophic microorganism in the bioreactor of claim 58, wherein the flexible boot has a constant interior diameter along the set length.

70. The method of producing phototrophic microorganism in the bioreactor of claim 58, wherein the flexible boot has a constant interior diameter along the set length and the constant interior diameter substantially equals an interior diameter of the port.

71. The method of producing phototrophic microorganism in the bioreactor of claim 58, wherein the thin-film wall has a thickness of between about 0.002-0.015 inches.

72. The method of producing phototrophic microorganism in the bioreactor of claim 58, wherein the connector produces a watertight seal with the thin-film wall of the reactor chamber and the port.

73. The method of producing phototrophic microorganism in the bioreactor of claim 58, wherein the flexible boot has an oval shape to receive the thin-film walls of the reactor chamber.
